(19) United States
(12) Patent Application Publication
(10) Pub. No.: US 2011/0039937 A1
(43) Pub. Date: Feb. 17, 2011

(54) NOVEL PROCESS FOR THE PREPARATION OF VORINOSTAT

(76) Inventors: Abhay Gaitonde, Maharashtra (IN); Bharati Choudhari, Maharashtra (IN)

Correspondence Address: FULBRIGHT & JAWORSKI L.L.P.;Attn: MN IP Docket, 600 Congress Avenue, Suite 2400, Austin, TX, 78701, US

(21) Appl. No.: 12/863,793

(22) PCT Filed: Feb. 6, 2009
(86) PCT No.: PCT/GB09/50117

§ 371 Date: Oct. 18, 2010

(30) Foreign Application Priority Data
Feb. 7, 2008 (IN) 220/KOL/2008

(51) Int. Cl. A61K 31/164; C07C 231/02; C07C 233/05; A61P 35/00

(52) U.S. Cl. 514/626; 564/138; 564/160

(57) ABSTRACT

The present invention relates to an improved process for the preparation of the active pharmaceutical ingredient, vorinostat. In particular it relates to an efficient process for the preparation of vorinostat of high purity without the requirement to isolate any synthetic intermediate compounds.

### CROSS-REFERENCE TO RELATED APPLICATION(s)

This application is a Section 371 National Stage Application of International No. PCT/GB2009/050117, filed 6 Feb. 2009 and published as WO 2009/098515 A1 on 13 Aug. 2009, which claims priority from the IN Patent Application No. 220/KOL/2008, filed 7 Feb. 2008, the contents of which are incorporated herein in their entirety for all purposes.

### FIELD OF THE INVENTION

The present invention relates to an improved process for the preparation of the active pharmaceutical ingredient, vorinostat. In particular it relates to an efficient process for the preparation of vorinostat of high purity without the requirement to isolate any synthetic intermediate compounds.

### BACKGROUND OF THE INVENTION

Vorinostat, also called suberoylanilide hydroxamic acid (SAHA) or N-hydroxy-N′-phenyl-octanediamide, is represented by the structural formula (I).

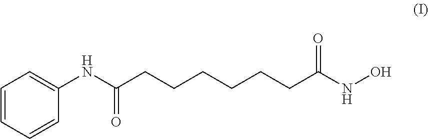

Vorinostat, a histone deacetylase (HDAC) inhibitor, is currently marketed for the treatment of cutaneous T cell lymphoma (CTCL), a type of skin cancer. It is used for treating patients having a tumor characterized by proliferation of neoplastic cells, as vorinostat is thought to be useful for selectively inducing terminal differentiation of neoplastic cells and thereby inhibiting proliferation of such cells under suitable conditions.

Processes for the preparation of vorinostat, and its form 1 crystalline polymorph, have been disclosed in patent applications US 2004/0122101 and WO 2006/127319. However, the disclosed processes, comprising the preparation of vorinostat from suberic acid, are a cumbersome three step process comprising the sequential steps of amidation of suberic acid with aniline, esterification of the mono-amide product with methanol, and finally reaction with hydroxylamine hydrochloride and sodium methoxide to afford vorinostat. This process is not very convenient as it involves elevated temperatures, lengthy reaction times and has a low overall yield of around 23%. In addition, the intermediate products and final product are not very pure and require exhaustive purification steps.

Four alternative processes to obtain vorinostat are reported in the U.S. Pat. No. 5,369,108 and these processes are illustrated in Schemes I, II, III and IV.

In Schemes I, III and IV, amide formation was reported by the reaction of suberoyl chloride, aniline and a third reactant. The third reactant was hydroxylamine hydrochloride, O-benzylhydroxylamine and O-(trimethylsilyl)hydroxylamine in Scheme I, III and IV respectively. The yield of vorinostat obtained in the three processes was almost the same (up to 35%) in each case.

In Scheme II, suberic acid monomethyl ester was converted into suberic acid monomethyl ester-monoacid chloride by treatment with oxaloyl chloride and dimethylformamide in benzene. The monomethyl ester-monoacid chloride thus formed was converted into the monoamide of suberic acid by treatment with aniline and subsequently potassium hydroxide. The suberic acid monoamide was treated with O-benzylhydroxylamine and 1,3-dicyclohexylcarbodiimide (DCC) in pyridine, followed by hydrogenolysis to afford vorinostat. The product yields were from 35% to 65%.

The processes illustrated in Schemes I, II, III and IV all require the use of column chromatography. The yields obtained in Schemes I, II, III and IV are 15-30%, 35-65%, 20-35% and 20-33% respectively.

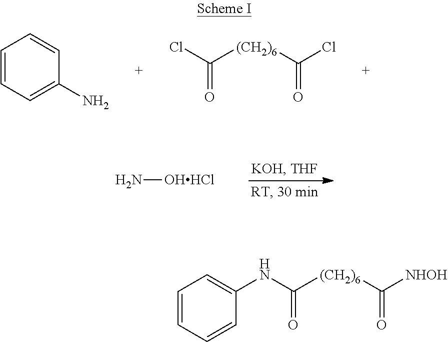

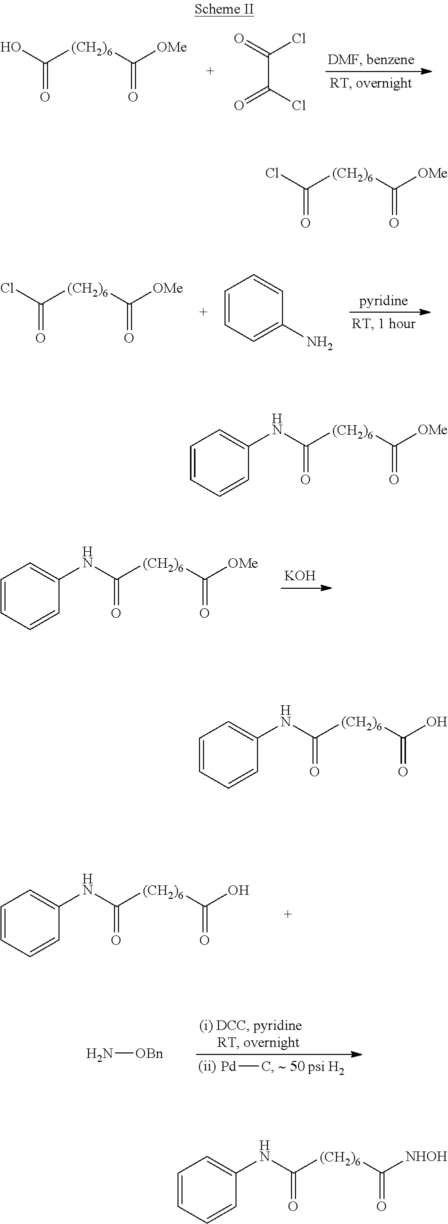

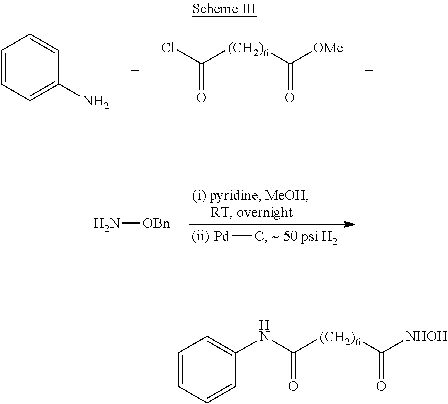

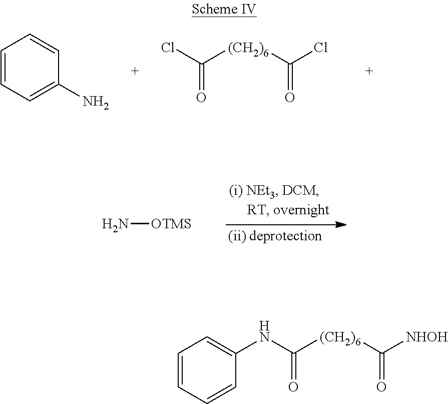

The major disadvantages of the processes disclosed in the prior art are as follows:
All schemes involve lengthy process steps to obtain vorinostat.
The reagents used in the processes can be very expensive. Consequently, the process is not cost effective enough for commercial manufacture.
The product is obtained only after column chromatography or extensive purification steps. This reduces the overall yield and puts severe restrictions on the feasibility of the process for scale-up to commercial production.
All the processes shown require the isolation and purification of all reaction intermediates.

In view of the importance acquired by vorinostat for the treatment of cancer, there is a great need for developing an alternative, relatively simple, economical and commercially feasible process for the synthesis of vorinostat with a commercially acceptable yield and high purity.

### SUMMARY OF THE INVENTION

The present inventors have surprisingly found that vorinostat can be prepared with very high purity employing a simple, efficient process starting with the readily available precursor suberic acid. The present inventors have also surprisingly observed that the process, particularly when carried out at reduced temperatures, avoids diamide formation and hence yields vorinostat of very high purity without the need for subsequent purification.

The present invention provides a simple, economical and commercially feasible process for the synthesis of vorinostat with a commercially acceptable yield and high purity.

The invention also provides a process for the synthesis of vorinostat wherein the synthetic intermediate compounds are not isolated.

The term vorinostat as used herein throughout the description and claims means vorinostat and/or any salt, solvate or polymorph thereof.

A first aspect of the present invention provides a process for the preparation of vorinostat, comprising the following steps:

(a) reaction of suberic acid and a haloformate;
(b) reaction of aniline with the product of step (a);
(c) reaction of a haloformate with the product of step (b);
(d) reaction of hydroxylamine with the product of step (c); and
(e) isolation of the product vorinostat.

Preferably, the haloformate in step (a) and step (c) is selected from the group comprising alkyl, alkenyl, alkynyl, aryl or arylalkyl haloformates. More preferably, the haloformate is selected from methyl, ethyl, benzyl or t-butyl haloformate. Preferably, the haloformate is a chloroformate, most preferably, methyl chloroformate.

Preferably, steps (a) and (c) are performed in the presence of a base. The base is preferably an organic base such as a trialkylamine such as triethylamine or a heterocyclic amine such as pyridine or 4-(dimethylamino)pyridine (DMAP). Preferably, the organic base is a trialkylamine, most preferably triethylamine.

Preferably, steps (a) to (d) are performed at a temperature below 20° C., preferably in a range between −5 to 15° C., more preferably between −5 to 10° C., more preferably between 0 to 10° C., and most preferably between 0 to 5° C.

Preferably steps (a) to (d) are performed in an organic solvent, wherein the organic solvent is preferably selected from the group comprising dimethyl sulfoxide, tetrahydrofuran, acetonitrile, dimethylformamide or dimethylacetamide. Most preferably, the organic solvent is tetrahydrofuran.

Preferably, the hydroxylamine in step (d) is present as a solution of hydroxylamine in an alcoholic solvent, wherein the alcoholic solvent is preferably selected from the group comprising alkyl, alkenyl or arylalkyl alcohols. More preferably, the alcoholic solvent is selected from the group comprising methanol, ethanol, isopropanol or butanol, and most preferably, the alcoholic solvent is methanol.

Preferably, the hydroxylamine solution is used at a temperature below 20° C., preferably in a range between −5 to 15° C., more preferably between −5 to 10° C., more preferably between 0 to 10° C., and most preferably between 0 to 5° C.

If required, the hydroxylamine can be used in the form of a suitable salt such as the hydrochloride salt.

Preferably the reaction products of steps (a) to (c) are not isolated and/or purified, making the sequence an efficient and convenient process for the preparation of vorinostat.

Preferably the process according to the first aspect of the invention is carried out without the use of chromatography.

Preferably the process is carried out in less than 5 hours, preferably less than 4 hours, preferably less than 3 hours, preferably less than 2 hours.

Preferably the process is carried out on an industrial scale, preferably to obtain vorinostat in batches of 100 g, 500 g, 1 kg, 5 kg, 10 kg, 25 kg or more.

Preferably the vorinostat is obtained in a yield of 30% or more, preferably 40% or more, from suberic acid.

Preferably the vorinostat obtained has a HPLC purity of 99% or more, preferably 99.5% or more, preferably 99.7% or more, preferably 99.8% or more, more preferably 99.9% or more.

A second aspect of the present invention provides substantially pure vorinostat. Preferably the vorinostat is suitable for use in medicine, preferably for treating or preventing cancer, preferably skin cancer, preferably cutaneous T cell lymphoma (CTCL).

A third aspect of the present invention provides substantially pure vorinostat as prepared by a process according to the first aspect of the invention. Preferably the vorinostat is suitable for use in medicine, preferably for treating or preventing cancer, preferably skin cancer, preferably cutaneous T cell lymphoma (CTCL).

A fourth aspect of the present invention provides a pharmaceutical composition comprising the vorinostat according to the second or third aspect of the invention.

A fifth aspect of the present invention provides use of the vorinostat according to the second or third aspect of the invention, or use of the pharmaceutical composition according to the fourth aspect of the invention, in the manufacture of a medicament for treating or preventing cancer, preferably skin cancer, more preferably cutaneous T cell lymphoma (CTCL).

A sixth aspect of the present invention provides a method of treating or preventing cancer, comprising administering to a patient in need thereof a therapeutically or prophylactically effective amount of the vorinostat according to the second or third aspect of the invention, or a therapeutically or prophylactically effective amount of the pharmaceutical composition according to the fourth aspect of the invention. Preferably the method according to the sixth aspect of the present invention is for treating or preventing skin cancer, more preferably cutaneous T cell lymphoma (CTCL). Preferably the patient is a mammal, preferably a human.

For the purposes of the present invention, an “alkyl” group is defined as a monovalent saturated hydrocarbon, which may be straight-chained or branched, or be or include cyclic groups. An alkyl group may optionally be substituted, and may optionally include one or more heteroatoms N, O or S in its carbon skeleton. Preferably an alkyl group is straight-chained or branched. Preferably an alkyl group is not substituted. Preferably an alkyl group does not include any heteroatoms in its carbon skeleton. Examples of alkyl groups are methyl, ethyl, n-propyl, i-propyl, n-butyl, i-butyl, t-butyl, n-pentyl, cyclopentyl, cyclohexyl and cycloheptyl groups. Preferably an alkyl group is a C₁₋₁₂ alkyl group, preferably a C₁₋₆ alkyl group. Preferably a cyclic alkyl group is a C₃₋₁₂ cyclic alkyl group, preferably a C₅₋₇ cyclic alkyl group.

An “alkenyl” group is defined as a monovalent hydrocarbon, which comprises at least one carbon-carbon double bond, which may be straight-chained or branched, or be or include cyclic groups. An alkenyl group may optionally be substituted, and may optionally include one or more heteroatoms N, O or S in its carbon skeleton. Preferably an alkenyl group is straight-chained or branched. Preferably an alkenyl group is not substituted. Preferably an alkenyl group does not include any heteroatoms in its carbon skeleton. Examples of alkenyl groups are vinyl, allyl, but-1-enyl, but-2-enyl, cyclohexenyl and cycloheptenyl groups. Preferably an alkenyl group is a C₂₋₁₂ alkenyl group, preferably a C₂₋₆ alkenyl group. Preferably a cyclic alkenyl group is a C₃₋₁₂ cyclic alkenyl group, preferably a C₅₋₇ cyclic alkenyl group.

An “alkynyl” group is defined as a monovalent hydrocarbon, which comprises at least one carbon-carbon triple bond, which may be straight-chained or branched, or be or include cyclic groups. An alkynyl group may optionally be substituted, and may optionally include one or more heteroatoms N, O or S in its carbon skeleton. Preferably an alkynyl group is straight-chained or branched. Preferably an alkynyl group is not substituted. Preferably an alkynyl group does not include any heteroatoms in its carbon skeleton. Examples of alkynyl groups are ethynyl, propargyl, but-1-ynyl and but-2-ynyl groups. Preferably an alkynyl group is a C₂₋₁₂ alkynyl group, preferably a C₂₋₆ alkynyl group. Preferably a cyclic alkynyl group is a C₃₋₁₂ cyclic alkynyl group, preferably a C₅₋₇ cyclic alkynyl group.

An “aryl” group is defined as a monovalent aromatic hydrocarbon. An aryl group may optionally be substituted, and may optionally include one or more heteroatoms N, O or S in its carbon skeleton. Preferably an aryl group is not substituted. Preferably an aryl group does not include any heteroatoms in its carbon skeleton. Examples of aryl groups are phenyl, naphthyl, anthracenyl, phenanthrenyl, thienyl and furyl groups. Preferably an aryl group is a C₄₋₁₄ aryl group, preferably a C₆₋₁₀ aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

An “alkoxy” group is defined as a —O-alkyl, —O-alkenyl, —O-alkynyl, —O-aryl, —O-arylalkyl, —O-arylalkenyl, —O-arylalkynyl, —O-alkylaryl, —O-alkenylaryl or —O-alkynylaryl group. Preferably an “alkoxy” group is a —O-alkyl or —O-aryl group. More preferably an “alkoxy” group is a —O-alkyl group.

A “halo” group is a fluoro, chloro, bromo or iodo group.

For the purposes of this invention, an optionally substituted group may be substituted with one or more of —F, —Cl, —Br, —I, —CF₃, —CCl₃, —CBr₃, —CI₃, —OH, —SH, —NH₂, —CN, —NO₂, —COOH, —R^{a}—O—R^{b}, —R^{a}—S—R^{b}, —R^{a}—N(R^{b})₂, —R^{a}—N(R^{b})₃⁺, —R^{a}—P(R^{b})₂, —R^{a}—Si(R^{b})₃, —R^{a}—CO—R^{b}, —R^{a}—CO—OR^{b}, —R^{a}O—CO—R^{b}, —R^{a}—CO—N(R^{b})₂, —R^{a}—NR^{b}—CO—R^{b}, —R^{a}O—CO—OR^{b}, —R^{a}O—CO—N(R^{b})₂, —R^{a}—NR^{b}—CO—OR^{b}, —R^{a}—NR^{b}—CO—N(R^{b})₂, —R^{a}—CS—R^{b} or —R^{b}. In this context, —R^{a}— is independently a chemical bond, or an unsubstituted C₁-C₁₀ alkylene, C₂-C₁₀ alkenylene or C₂-C₁₀ alkynylene group. —R^{b} is independently hydrogen, or an unsubstituted C₁-C₆ alkyl or C₆-C₁₀ aryl group. Optional substituent(s) are not taken into account when calculating the total number of carbon atoms in the parent group substituted with the optional substituent(s). Preferably a substituted group comprises 1, 2 or 3 substituents, more preferably 1 or 2 substituents, and even more preferably 1 substituent.

For the purposes of the present invention, a compound is “substantially pure” if it comprises less than 1% impurity by HPLC, preferably less than 0.5%, preferably less than 0.3%, preferably less than 0.2%, preferably less than 0.1%.

### DETAILED DESCRIPTION OF THE INVENTION

The present invention provides an efficient and economical synthesis of vorinostat, starting from the readily available suberic acid, and affords the product with very high purity.

The present inventors have surprisingly observed that vorinostat can be prepared with commercially acceptable yield employing an extremely convenient process starting with suberic acid and methyl chloroformate, without isolation and/or purification of the synthetic intermediate compounds.

The present inventors have, surprisingly, also found that the process, particularly when carried out at reduced temperature, yields substantially pure vorinostat. Vorinostat is “substantially pure” if it comprises less than 1% impurity by HPLC, preferably less than 0.5%, preferably less than 0.3%, preferably less than 0.2%, preferably less than 0.1%.

The present inventors have surprisingly found that the process according to the first aspect of the invention includes the advantages of large reductions in reaction time as compared to the prior art processes, very easy and efficient purification techniques, and very high purity (>99% by HPLC).

In a preferred embodiment of the present invention, the synthetic intermediate products are not isolated and/or purified. However, as part of the present invention, the synthetic intermediates may be isolated and/or purified if so desired.

A preferred embodiment of the first aspect of the invention is illustrated in Scheme V.

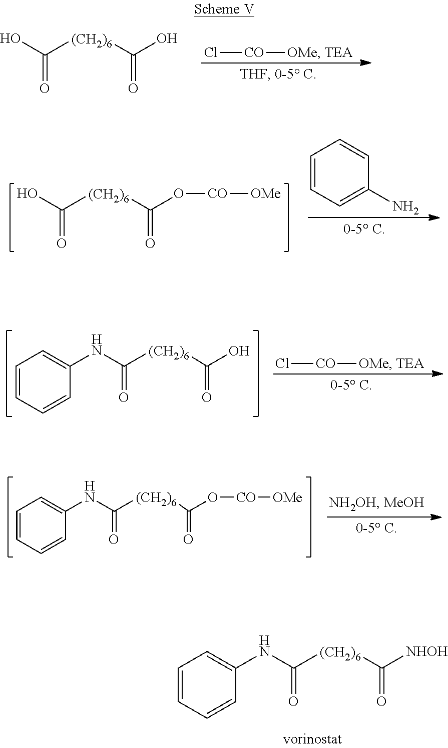

A preferred embodiment of the present invention comprises the following steps:

(a) a mixture of suberic acid and methyl chloroformate is dissolved in an organic solvent in the presence of triethylamine at 0-5° C.;
(b) aniline is added to the product of step (a) at 0-5° C.;
(c) methyl chloroformate is added to the reaction mixture of step (b) in the presence of triethylamine at 0-5° C.;
(d) the reaction mixture obtained in step (c) is added to a solution of cooled, freshly prepared hydroxylamine in methanol.

A typical work up procedure to isolate substantially pure vorinostat comprises the following steps:

(e) the reaction solvent is removed from the reaction mixture of step (d) under vacuum preferably at about 40° C.;
(f) methylene dichloride is added to the residue of step (e) and the organic solution obtained is washed with water and dried preferably over anhydrous sodium sulfate;
(g) the methylene dichloride is removed under vacuum preferably at about 40° C. and acetonitrile is added to the residue to isolate a solid product which is filtered under vacuum;
(h) the solid product from step (g), vorinostat, is dried under vacuum preferably at about 60° C.

The above sequence is a very short, efficient process for the production of substantially pure vorinostat with no requirement for cumbersome purification techniques. Therefore the process of the present invention is extremely suitable for commercial production of substantially pure vorinostat.

The pharmaceutical composition according to the fourth aspect of the present invention can be a solution or suspension, but is preferably a solid oral dosage form. Preferred solid oral dosage forms in accordance with the invention include tablets, capsules and the like which, optionally, may be coated if desired. Tablets can be prepared by conventional techniques, including direct compression, wet granulation and dry granulation. Capsules are generally formed from a gelatine material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant and optionally further comprises at least one excipient selected from colouring agents, adsorbents, surfactants, film formers and plasticizers.

If the solid pharmaceutical formulation is in the form of coated tablets, the coating may be prepared from at least one film-former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

The following paragraphs enumerated consecutively from 1 through 47 provide for various aspects of the present invention. In one embodiment, the present invention provides:

1. A process for the preparation of vorinostat, comprising the following steps:
(a) reaction of suberic acid and a haloformate;
(b) reaction of aniline with the product of step (a);
(c) reaction of a haloformate with the product of step (b);
(d) reaction of hydroxylamine with the product of step (c); and
(e) isolation of the product vorinostat.
2. A process according to paragraph 1, wherein the haloformate in step (a) and step (c) is independently selected from the group comprising alkyl, alkenyl, alkynyl, aryl or arylalkyl haloformates.
3. A process according to paragraph 2, wherein the haloformate is independently selected from methyl, ethyl, benzyl or t-butyl haloformate.
4. A process according to any one of the preceding paragraphs, wherein the haloformate in step (a) and step (c) is a chloroformate.
5. A process according to paragraph 4, wherein the chloroformate is methyl chloroformate.
6. A process according to any one of the preceding paragraphs, wherein steps (a) and (c) are performed in the presence of a base.
7. A process according to paragraph 6, wherein the base is an organic base.
8. A process according to paragraph 7, wherein the organic base is a trialkylamine.
9. A process according to paragraph 8, wherein the organic base is triethylamine.
10. A process according to any one of the preceding paragraphs, wherein steps (a) to (d) are performed at a temperature below 20° C.
11. A process according to paragraph 10, wherein the temperature is between −5 to 15° C.
12. A process according to paragraph 11, wherein the temperature is between −5 to 10° C.
13. A process according to paragraph 12, wherein the temperature is between 0 to 10° C.
14. A process according to paragraph 13, wherein the temperature is between 0 to 5° C.
15. A process according to any one of the preceding paragraphs, wherein steps (a) to (d) are performed in an organic solvent.
16. A process according to paragraph 15, wherein the organic solvent is selected from the group comprising dimethyl sulfoxide, tetrahydrofuran, acetonitrile, dimethylformamide or dimethylacetamide.
17. A process according to paragraph 16, wherein the organic solvent is tetrahydrofuran.
18. A process according to any one of the preceding paragraphs, wherein the hydroxylamine in step (d) is present as a solution of hydroxylamine in an alcoholic solvent.
19. A process according to paragraph 18, wherein the alcoholic solvent is selected from the group comprising alkyl, alkenyl or arylalkyl alcohols.
20. A process according to paragraph 19, wherein the alcoholic solvent is selected from the group comprising methanol, ethanol, isopropanol or butanol.
21. A process according to paragraph 20, wherein the alcoholic solvent is methanol.
22. A process according to any one of paragraphs 18 to 21, wherein the hydroxylamine solution is used at a temperature below 20° C.
23. A process according to paragraph 22, wherein the temperature is between −5 to 15° C.
24. A process according to paragraph 23, wherein the temperature is between −5 to 10° C.
25. A process according to paragraph 24, wherein the temperature is between 0 to 10° C.
26. A process according to paragraph 25, wherein the temperature is between 0 to 5° C.
27. A process according to any one of the preceding paragraphs, wherein the reaction products of steps (a) to (c) are not isolated and/or purified.
28. A process according to any one of the preceding paragraphs, wherein the process is carried out without the use of chromatography.
29. A process according to any one of the preceding paragraphs, wherein the process is carried out in less than 5 hours.
30. A process according to any one of the preceding paragraphs, wherein the process is carried out on an industrial scale.
31. A process according to any one of the preceding paragraphs, wherein the vorinostat is obtained in a yield of 30% or more from suberic acid.
32. A process according to any one of the preceding paragraphs, wherein the vorinostat obtained has a HPLC purity of 99% or more.
33. Substantially pure vorinostat.
34. Substantially pure vorinostat as prepared by a process according to any one of paragraphs 1 to 32.
35. Vorinostat according to paragraph 33 or 34, for use in medicine.
36. Vorinostat according to paragraph 35, for treating or preventing cancer.
37. Vorinostat according to paragraph 36, for treating or preventing skin cancer.
38. Vorinostat according to paragraph 37, for treating or preventing cutaneous T cell lymphoma (CTCL).
39. A pharmaceutical composition comprising vorinostat according to any one of paragraphs 33 to 38.
40. Use of vorinostat according to any one of paragraphs 33 to 38, or use of the pharmaceutical composition according to paragraph 39, in the manufacture of a medicament for treating or preventing cancer.
41. The use according to paragraph 40, for treating or preventing skin cancer.
42. The use according to paragraph 41, for treating or preventing cutaneous T cell lymphoma (CTCL).
43. A method of treating or preventing cancer, comprising administering to a patient in need thereof a therapeutically or prophylactically effective amount of vorinostat according to any one of paragraphs 33 to 38, or a therapeutically or prophylactically effective amount of the pharmaceutical composition according to paragraph 39.
44. The method according to paragraph 43, for treating or preventing skin cancer.
45. The method according to paragraph 44, for treating or preventing cutaneous T cell lymphoma (CTCL).
46. The method according to any one of paragraphs 43 to 45, wherein the patient is a mammal.
47. The method according to paragraph 46, wherein the patient is a human.

The details of the invention, its objects and advantages are illustrated below in greater detail by a non-limiting example.

### Example

### Vorinostat

Suberic acid (1.0 eq) was dissolved in tetrahydrofuran (15 vol) and the clear solution was chilled to 0-5° C. Methyl chloroformate (1.1 eq) and triethylamine (1.1 eq) were added to the solution at the same temperature and the mixture was stirred for 15 minutes. The triethylamine.HCl salt formed was filtered off, then aniline (1 eq) was added to the reaction mixture at 0-5° C. and stirring was continued for 15 minutes. Methyl chloroformate (1.1 eq) and triethylamine (1.1 eq) were added to the clear solution and stirring was continued for a further 15 minutes at 0-5° C. This chilled reaction mixture was added to a freshly prepared hydroxylamine solution in methanol (*see below) chilled to 0-5° C. and stirred for 15 minutes at 0-5° C. The solvent was removed under vacuum at 40° C. and the residue obtained was taken in methylene dichloride and the organic solution was washed with water and dried over anhydrous sodium sulfate. Methylene dichloride was removed under vacuum at 40° C. and acetonitrile was added to the residue. This mixture was stirred for 15 minutes before the solid was filtered under vacuum and dried under vacuum at 60° C. to afford the product as a white solid. Molar yield=35-41%; HPLC purity=99.90%.

¹H-NMR (DMSO-d₆): 1.27 (m, 4H, 2×—CH₂—), 1.53 (m, 4H, 2×—CH₂—), 1.94 (t, J=7.3 Hz, 2H, —CH₂—), 2.29 (t, J=7.4 Hz, 2H, —CH₂—), 7.03 (t, J=7.35 Hz, 1H, aromatic para position), 7.27 (t, J=7.90 Hz, 2H, aromatic meta position), 7.58 (t, J=7.65 Hz, 2H, aromatic ortho position), 8.66 (s, 1H, —OH, D₂O exchangeable), 9.85 (s, 1H, amide —NH—, D₂O exchangeable), 10.33 (s, 1H, —NH—OH, D₂O exchangeable).

¹³C-NMR (DMSO-d₆): 25.04 (2C, 2×—CH₂—), 28.43 (2C, 2×—CH₂—), 32.24 (1C, —CH₂—), 36.34 (1C, —CH₂—), 119.01 (2C, Ar—C), 122.96 (1C, Ar—C), 128.68 (2C, Ar—C), 139.24 (1C, Ar—C, ═CNH—), 169.23 (1C, —CO—), 171.50 (1C, —CO—).

Preparation of Hydroxylamine Solution:

Potassium hydroxide (1.1 eq) was added to methanol (8 vol) and the solution was chilled to 0-5° C. Similarly hydroxylamine hydrochloride (1.1 eq) was added to methanol (8 vol) and chilled to 0-5° C. The chilled amine solution was added to the chilled alkali solution and stirred for 15 minutes at 0-5° C. The white potassium chloride salt was filtered off and the filtrate was used as such.

1-52. (canceled)

53. A process for the preparation of vorinostat, comprising the following steps:
(a) reaction of suberic acid and a haloformate;
(b) reaction of aniline with the product of step (a);
(c) reaction of a haloformate with the product of step (b);
(d) reaction of hydroxylamine with the product of step (c); and
(e) isolation of the product vorinostat.

54. A process according to claim 53, wherein the haloformate in step (a) and step (c) is:
(i) independently selected from the group comprising alkyl, alkenyl, alkynyl, aryl or arylalkyl haloformates; and/or
(ii) independently selected from methyl, ethyl, benzyl or t-butyl haloformate; and/or
(iii) a chloroformate; and/or
(iv) methyl chloroformate.

55. A process according to claim 53, wherein steps (a) and (c) are performed in the presence of:
(i) a base; and/or
(ii) an organic base; and/or
(iii) a trialkylamine; and/or
(iv) triethylamine.

56. A process according to claim 53, wherein steps (a) to (d) are performed at a temperature:
(i) below 20° C.; and/or
(ii) between −5 to 15° C.; and/or
(iii) between −5 to 10° C.; and/or
(iv) between 0 to 10° C.; and/or
(v) between 0 to 5° C.

57. A process according to claim 53, wherein steps (a) to (d) are performed in:
(i) an organic solvent; and/or
(ii) an organic solvent selected from the group comprising dimethyl sulfoxide, tetrahydrofuran, acetonitrile, dimethylformamide or dimethylacetamide; and/or
(iii) tetrahydrofuran.

58. A process according to claim 53, wherein the hydroxylamine in step (d) is present as a solution of hydroxylamine:
(i) in an alcoholic solvent; and/or
(ii) in an alcoholic solvent selected from the group comprising alkyl, alkenyl or arylalkyl alcohols; and/or
(iii) in an alcoholic solvent selected from the group comprising methanol, ethanol, isopropanol or butanol; and/or
(iv) in methanol.

59. A process according to claim 53, wherein the hydroxylamine in step (d) is present as a solution of hydroxylamine in an alcoholic solvent, and wherein the hydroxylamine solution is used at a temperature:
(i) below 20° C.; and/or
(ii) between −5 to 15° C.; and/or
(iii) between −5 to 10° C.; and/or
(iv) between 0 to 10° C.; and/or
(v) between 0 to 5° C.

60. A process according to claim 53, wherein:
(i) the reaction products of steps (a) to (c) are not isolated and/or purified; and/or
(ii) the process is carried out without the use of chromatography; and/or
(iii) the process is carried out in less than 5 hours; and/or
(iv) the process is carried out on an industrial scale; and/or
(v) the vorinostat is obtained in a yield of 30% or more from suberic acid; and/or
(vi) the vorinostat obtained has a HPLC purity of 99% or more.

61. Substantially pure vorinostat.

62. Substantially pure vorinostat as prepared by a process according to claim 53.

63. Vorinostat according to claim 61, for:
(i) use in medicine; and/or
(ii) treating or preventing cancer; and/or
(iii) treating or preventing skin cancer; and/or
(iv) treating or preventing cutaneous T cell lymphoma (CTCL).

64. A pharmaceutical composition comprising vorinostat according to claim 61.

65. A method of treating or preventing cancer, comprising administering to a patient in need thereof a therapeutically or prophylactically effective amount of vorinostat according to claim 61.

66. The method according to claim 65, for:
(i) treating or preventing skin cancer; and/or
(ii) treating or preventing cutaneous T cell lymphoma (CTCL).

67. The method according to claim 65, wherein the patient is:
(i) a mammal; and/or
(ii) a human.

68. A method of treating or preventing cancer, comprising administering to a patient in need thereof a therapeutically or prophylactically effective amount of the pharmaceutical composition according to claim 64.

69. The method according to claim 68, for:
(i) treating or preventing skin cancer; and/or
(ii) treating or preventing cutaneous T cell lymphoma (CTCL).

70. The method according to claim 68, wherein the patient is:
(i) a mammal; and/or
(ii) a human.
